(19) United States
(12) Patent Application Publication
(10) Pub. No.: US 2018/0326182 A1
(43) Pub. Date: Nov. 15, 2018

(54) STEERING ASSEMBLY INCLUDING STEERING RING FOR NAVIGATION OF CATHETER

(71) Applicant: CRYTERION MEDICAL, INC., Carlsbad, CA (US)

(72) Inventor: Eugene J. Jung, JR., San Diego, CA (US)

(73) Assignee: Cryterion Medical, Inc.

(21) Appl. No.: 15/926,938

(22) Filed: Mar. 20, 2018

Related U.S. Application Data
(60) Provisional application No. 62/503,908, filed on May 9, 2017.

(51) Int. Cl. A61M 25/01; A61B 18/02

(52) U.S. Cl. CPC A61M 25/0147; A61M 25/0113; A61B 2018/0022; A61M 2025/015; A61M 25/0136; A61B 18/02

(57) ABSTRACT

A steering assembly for steering, navigating, articulating and/or positioning a catheter includes a steering ring having a first edge surface, a second edge surface and a side surface. The steering ring includes one or more notches formed within the side surface. The notches are spaced apart and extend to one of the first edge surface and the second edge surface. The steering ring can also include one or more apertures formed within the side surface. In one embodiment, the steering ring can include one or more lattice structures formed within the side surface. In certain embodiments, the lattice structures can form at least approximately 10%, 25% and/or 50% of the side surface.

### RELATED APPLICATION

This application claims priority on U.S. Provisional Application Ser. No. 62/503,908 filed on May 9, 2017 and entitled “STEERING ASSEMBLY INCLUDING STEERING RING FOR NAVIGATION OF CATHETER.” As far as permitted, the contents of U.S. Provisional Application Ser. No. 62/503,908 are incorporated herein by reference.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning a portion, such as a distal tip, of an energy delivery catheter adjacent to diseased or targeted tissue in the heart. One form of energy that is used to ablate diseased heart tissue includes cryogenics (also referred to herein as “cryoablation”). During this procedure, the most distal (i.e., furthest from the operator) portion of the catheter, and often at the distal tip of the device, is positioned adjacent to targeted cardiac tissue, at which time energy is delivered to create tissue necrosis, rendering the ablated tissue incapable of conducting electrical signals.

Atrial fibrillation is one of the most common arrhythmias treated using cryoablation. In the earliest stages of the disease, paroxysmal atrial fibrillation, the treatment strategy involves isolating the pulmonary veins from the left atrial chamber. Recently, the use of techniques known as “balloon cryotherapy” catheter procedures to treat atrial fibrillation have increased. During the balloon cryotherapy procedure, a refrigerant or cryogenic fluid (such as nitrous oxide, or any other suitable fluid) is delivered under pressure to an interior of one or more cryogenic balloons which are positioned adjacent to or against the targeted cardiac tissue. Using this method, the extremely frigid cryogenic fluid causes necrosis of the targeted cardiac tissue, thereby rendering the ablated tissue incapable of conducting unwanted electrical signals.

During cryoablation procedures, the distal tip of the catheter is designed to reach tissue within the patient's heart. In order to reach various locations within the heart, the procedure requires that the catheter be carefully steered or navigated through the patient's body, particularly the patient's vascular path. Navigation of the catheter is generally performed with the use of one or more pull wires attached to a steering ring connected at or near the distal tip of the catheter. Specifically, applying force on the pull wires causes the distal tip of the catheter to deflect or articulate, allowing the catheter to be steered, navigated and/or ultimately positioned advantageously in a region of interest for the cryoablation procedure. The wide ranging forces applied on the pull wires to steer or navigate the catheter can often result in stresses or forces that may affect or ultimately affect the strength or integrity of the steering ring and/or the connection between the steering ring and the catheter. Due to these additional forces, it is not uncommon for steering rings to fracture or become detached from the catheter. Any fracture or detachment during the cryoablation procedure would not only interrupt the procedure, but it could also be injurious to the patient.

### SUMMARY

The present invention is directed toward a steering assembly for a cryogenic balloon catheter system. In one embodiment, the steering assembly includes a steering ring. The steering ring can include a first edge surface, a second edge surface and a side surface. Further, the steering ring can include a first notch formed within the side surface. In certain embodiments, the first notch can extend to one of the first edge surface and the second edge surface.

In various embodiments, the steering ring can further include a second notch. The second notch can be formed within the side surface. Further, the second notch is spaced apart from the first notch. In one embodiment, the second notch can be spaced apart from the first notch by approximately 180 degrees along the side surface. In some embodiments, the second notch can extend to one of the first edge surface and the second edge surface. In other embodiments, the first notch and the second notch can both extend to the first edge surface.

In alternative embodiments, the steering ring can include an aperture. The aperture can be formed within the side surface. The aperture is spaced apart from the first notch. In one embodiment, the aperture can be spaced apart from the first notch by approximately 180 degrees along the side surface.

In certain embodiments, the steering assembly can further include one or more pull wires. In such embodiments, the one or more pull wires can be connected to the steering ring.

The present invention is also directed toward a steering assembly for a cryogenic balloon catheter system. In another embodiment, the steering assembly can include a steering ring. The steering ring can include a first edge surface, a second edge surface and a side surface. However, in this embodiment, the steering ring can include a first lattice structure. The first lattice structure can be formed within the side surface. In certain embodiments, the first lattice structure formed within the side surface can extend to one of the first edge surface and the second edge surface.

In various embodiments, the steering ring can include a second lattice structure. The second lattice structure can be formed within the side surface. The second lattice structure is spaced apart from the first lattice structure. In one embodiment, the second lattice structure can be positioned approximately 180 degrees along the side surface from the first lattice structure. In some embodiments, the second lattice structure formed within the side surface can extend to one of the first edge surface and the second edge surface. In other embodiments, the first lattice structure and the second lattice structure both can extend to the first edge surface.

In certain embodiments, at least one of the first lattice structure and second lattice structure formed within the side surface can comprise at least approximately 10 or 25 percent of the side surface. In other embodiments, each of the first lattice structure and the second lattice structure can comprise at least approximately 10 or 25 percent of the side surface. In one embodiment, the first lattice structure formed within the side surface can comprise at least approximately 50 percent of the side surface.

The present invention is also directed toward a steering assembly for a cryogenic balloon catheter system. In certain embodiments, the steering assembly can include one or more pull wires and a steering ring. In such embodiments, the steering ring can be connected to the one or more pull wires. The steering ring can also include a first edge surface, a second edge surface and a side surface. In some embodiments, the steering ring can further include a first notch and a second notch. The first notch is spaced apart from the second notch. Each of the first notch and the second notch can be formed within the side surface. In certain embodiments, the first notch and the second notch can extend to one of the first edge surface and the second edge surface.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic side view of a patient and one embodiment of a cryogenic balloon catheter system having features of the present invention;

FIG. 2 is a simplified side view of an embodiment of a portion of the cryogenic balloon catheter system including one embodiment of a steering assembly;

FIG. 3 is a perspective view of another embodiment of a portion of the steering assembly including one embodiment of a steering ring;

FIG. 4 is a perspective view of still another embodiment of a portion of the steering assembly including another embodiment of the steering ring; and

FIG. 5 is a perspective view of yet another embodiment of a portion of the steering assembly including still another embodiment of the steering ring.

### DESCRIPTION

Embodiments of the present invention are described herein in the context of a steering assembly including a steering ring for navigation of a catheter. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy are used to ablate diseased heart tissue. These can include radio frequency, ultrasound and laser energy, to name a few. The present invention is intended to be effective with any or all of these forms of energy.

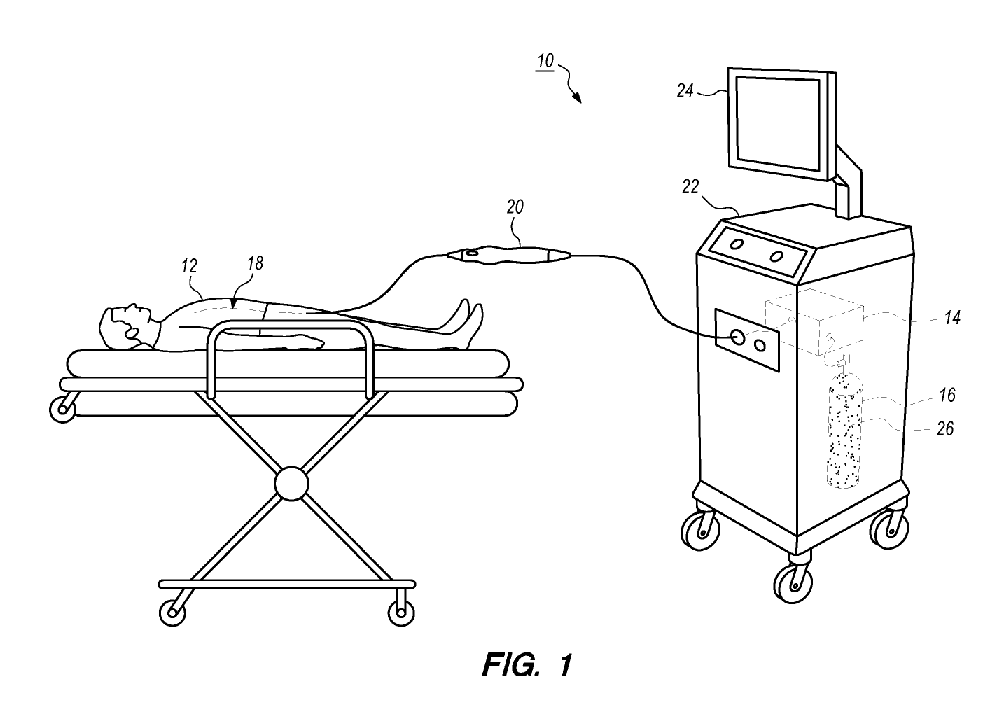

FIG. 1 is a schematic side view of one embodiment of a cryogenic balloon catheter system 10 (also sometimes referred to herein as a “catheter system”) for use with a patient 12, which can be a human being or an animal. Although the catheter system 10 is specifically described herein with respect to a cryogenic balloon catheter system, it is understood and appreciated that other types of catheter systems and/or ablation systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of the cryogenic balloon catheter system is not intended to be limiting in any manner.

The design of the catheter system 10 can be varied. In certain embodiments, such as the embodiment illustrated in FIG. 1, the catheter system 10 can include one or more of a control system 14, a fluid source 16, a balloon catheter 18, a handle assembly 20, a control console 22 and a graphical display 24. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional components than those specifically illustrated and described herein.

In various embodiments, the control system 14 is configured to monitor and control the various processes of a cryoablation procedure. More specifically, the control system 14 can control release and/or retrieval of a cryogenic fluid 26 to and/or from the balloon catheter 18. In certain embodiments, the control system 14 can control various structures described herein that are responsible for maintaining and/or adjusting a flow rate and/or a fluid pressure of the cryogenic fluid 26 that is released to the balloon catheter 18 during the cryoablation procedure. In such embodiments, the catheter system 10 delivers ablative energy in the form of cryogenic fluid 26 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18 described herein. Further, or in the alternative, the control system 14 can receive electrical signals, data and/or other information (hereinafter sometimes referred to as “sensor output”) from various structures within the catheter system 10. In some embodiments, the control system 14 can assimilate and/or integrate the sensor output, and/or any other data or information received from any structure within the catheter system 10. Additionally, or in the alternative, the control system 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the control system 14 during the cryoablation procedure. The type of cryogenic fluid 26 that is used during the cryoablation procedure can vary. In one non-exclusive embodiment, the cryogenic fluid 26 can include liquid nitrous oxide. In another non-exclusive embodiment, the cryogenic fluid 26 can include liquid nitrogen. However, any other suitable cryogenic fluid 26 can be used.

The design of the balloon catheter 18 can be varied to suit the specific design requirements of the catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a health care professional (also sometimes referred to herein as an “operator”). As used herein, health care professional and/or operator can include a physician, a physician's assistant, a nurse and/or any other suitable person or individual. In certain embodiments, the balloon catheter 18 is positioned within the body of the patient 12 utilizing at least a portion of the sensor output received from the balloon catheter 18. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the operator to operate, position and/or control the balloon catheter 18. The design and specific features of the handle assembly 20 can vary to suit the specific design requirements of the catheter system 10. In the embodiment illustrated in FIG. 1, the handle assembly 20 is separate from, but in electrical and/or fluid communication with the control system 14, the fluid source 16 and/or the graphical display 24. In some embodiments, the handle assembly 20 can integrate and/or include at least a portion of the control system 14 within an interior of the handle assembly 20. It is understood that the handle assembly 20 can include additional components than those specifically illustrated and described herein.

In the embodiment illustrated in FIG. 1, the control console 22 includes at least a portion of the control system 14, the fluid source 16 and the graphical display 24. However, in alternative embodiments, the control console 22 can contain additional structures not shown or described herein. Still alternatively, the control console 22 may not include various structures that are illustrated within the control console 22 in FIG. 1. For example, in one embodiment, the control console 22 does not include the graphical display 24.

In various embodiments, the graphical display 24 is electrically connected to the control system 14. Additionally, the graphical display 24 provides the operator of the catheter system 10 with information that can be used before, during and after the cryoablation procedure. For example, the graphical display 24 can provide the operator with information based on the sensor output, and any other relevant information that can be used before, during and after the cryoablation procedure. The specifics of the graphical display 24 can vary depending upon the design requirements of the catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time. Further, in various embodiments, the graphical display 24 can include one or more colors, different sizes, varying brightness, etc., that may act as alerts to the operator. Additionally, or in the alternative, the graphical display 24 can provide audio data or information to the operator.

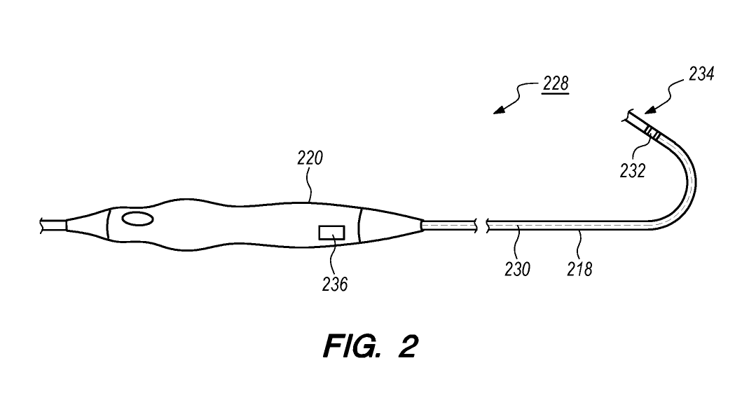

FIG. 2 is a simplified side view of an embodiment of a portion of the catheter system 10 (illustrated in FIG. 1) including one embodiment of a steering assembly 228. In various embodiments, the steering assembly 228 enables the operator to steer, navigate, articulate and/or adjust the positioning of the balloon catheter 218 during cryoablation procedures. The design of the steering assembly 228 can be varied. In certain embodiments, such as the embodiment illustrated in FIG. 2, the steering assembly 228 includes at least a portion of the balloon catheter 218 and the handle assembly 220. It is recognized that the simplified steering assembly 228 illustrated in FIG. 2 is for representative purposes only, and that any suitable steering assembly 228 can be used during cryoablation procedures. The steering assembly 228 can include additional components or fewer components than those specifically illustrated and described herein. Moreover, it is understood that the steering assembly 228 can include such additional components, or omit certain components. The specific components and operation of the steering assembly 228 will be described in greater detail herein below.

In certain embodiments, the balloon catheter 218 can include one or more pull wires 230 and a steering ring 232, wherein the balloon catheter 218 may be steered or articulated by the manipulation or maneuvering of one or more pull wires 230. Alternatively, the balloon catheter 218 can include additional components or fewer components than those specifically illustrated and described herein. In various embodiments, the steering assembly 228 can include the one or more pull wires 230 and/or the steering ring 232.

The pull wire(s) 230 can extend from the handle assembly 220 to the steering ring 232, wherein the steering ring 232 is located or positioned at a distal end 234 of the balloon catheter 218. In various embodiments, portions of the pull wire(s) 230 can be located within an interior of the balloon catheter 218, including a dedicated lumen (not shown) within the catheter shaft wall (not shown). In one embodiment, the pull wire(s) 230 may be sheathed in a Teflon liner for lubricity. More particularly, in certain embodiments, the pull wire(s) 230 can be coupled to the handle assembly 220 which can allow the pull wire(s) 230 to be manipulated or maneuvered by the operator to steer, navigate, articulate and/or position the balloon catheter 218 during cryoablation procedures. The pull wire(s) 230 may be coupled to the handle assembly 220 in any suitable manner.

In certain embodiments, the pull wire(s) 230 may have a circular cross-section. In alternative embodiments, the pull wire(s) 230 may have the cross-section of any other suitable shape or design. Further, the pull wire(s) 230 may be made from any suitable material or materials.

The steering ring 232 can be located at or near the distal end 234 of the balloon catheter 218. In various embodiments, the pull wire(s) 230 can be coupled to the steering ring 232 to enable the operator to steer, navigate, articulate and/or position the balloon catheter 218. The pull wire(s) 230 may be coupled to the steering ring 232 in any suitable manner, including weld or solder joint, adhesive or bonding material, as certain non-exclusive examples. In one embodiment, the steering ring 232 can also be connected to the balloon catheter 218 within an interior (not shown) of the balloon catheter 218 with the use of an adhesive or a thermal bonding technique, as non-exclusive examples. In other embodiments, the steering ring 232 can also be connected to the interior of the balloon catheter 218 with the use of any other suitable bonding material. Alternatively, the steering ring 232 may be connected to the interior of the balloon catheter 218 in any other suitable manner which allows the operator to steer, navigate, articulate and/or position the balloon catheter 218 to a desired location, e.g., adjacent to targeted cardiac tissue. Further, the steering ring 232 may be made from any suitable material or materials, such as stainless steel, for example. The specific configuration and/or design of the steering ring 232 will be described in greater detail herein below.

In the embodiment illustrated in FIG. 2, the steering ring 232 is located at or near the distal end 234 of the balloon catheter 218. Accordingly, when the operator manipulates or maneuvers the pull wire(s) 230, i.e., applies a force in any direction, the balloon catheter 218 may then be steered, navigated and/or articulated in the direction of the force being exerted by the pull wire(s) 230 on the steering ring 232.

The handle assembly 220 enables the operator to operate, steer, position and/or control the balloon catheter 218. The design and specific features of the handle assembly 220 can vary. In some embodiments, the handle assembly 220 can include a controller 236 within an interior of the handle assembly 220. In such embodiments, the controller 236 may be configured to operate, steer, position and/or control the balloon catheter 218. Specifically, the controller 236 can control positioning of portions of the balloon catheter 218, and/or can control any other suitable functions of the balloon catheter 218, including the manipulating or maneuvering of the pull wire(s) 230 and/or the forces being applied to the steering ring 232. It is understood that the handle assembly 220 can include additional components than those specifically illustrated and described herein. In certain embodiments, the steering assembly 228 can include the controller 236.

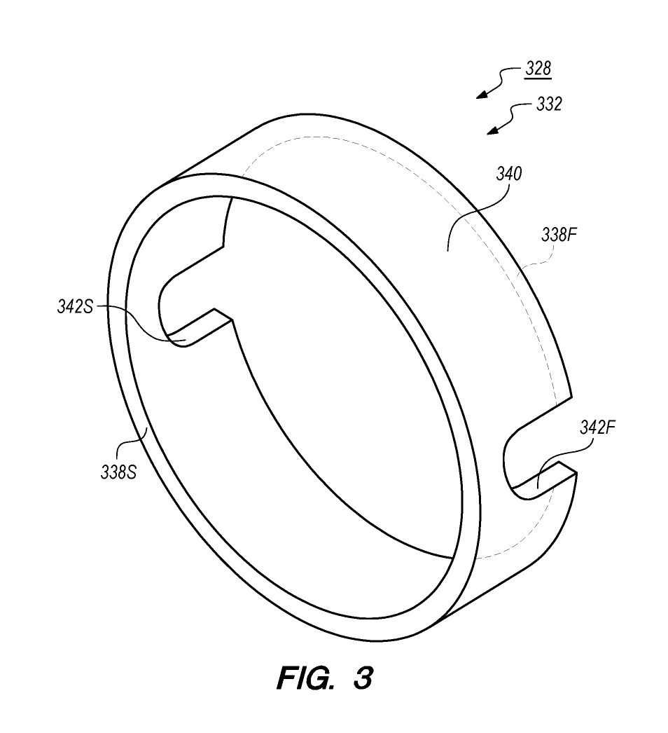

FIG. 3 is a perspective view of another embodiment of a portion of the steering assembly 328 including one embodiment of the steering ring 332. In certain embodiments, as shown in FIG. 3, the steering ring 332 can include a first edge surface 338F, a second edge surface 338S, a side surface 340, a first notch 342F and a second notch 342S. It is recognized that the terms “first edge surface 338F” and “second edge surface 338S” can be used interchangeably. It is further recognized that the terms “first notch 342F” and “second notch 342S” can be used interchangeably. In other words, either notch 342F, 342S, can be the first notch 342F or the second notch 342S.

The first edge surface 338F and the second edge surface 338S may form an edge of the steering ring 332 and may be of varying thicknesses. The side surface 340 extends between the first edge surface 338F and the second edge surface 338S and may form a continuous area around the circumference of the steering ring 332. The side surface 340 can include a varying width. While FIG. 3 shows two notches 342F, 342S, it should be appreciated that the steering ring 332 may include any suitable number of notches 342F, 342S. In other words, the steering ring 332 can include a plurality of notches 342F, 342S, i.e., first notch, second notch, third notch, etc. Further, it is understood that one or more pull wires 230 (illustrated in FIG. 2) may be coupled to the steering ring 332. For ease in understanding, the pull wire(s) 230 and coupling means have been omitted from FIGS. 3-5.

The steering ring 332 can be bonded, adhered and/or connected to the interior (not shown) of the balloon catheter 218 (illustrated in FIG. 2) in any suitable manner, i.e., thermal bonding, adhesive, bonding material, etc., which may allow the operator to steer, navigate, articulate and/or position the balloon catheter 218 to the desired location. In various embodiments, the notches 342F, 342S, may allow catheter shaft material, adhesive or bonding material, or other material, to run into, through and/or around each notch 342F, 342S, creating a more secure, durable and/or resilient bond or connection between the balloon catheter 218 and steering ring 332. The improved bond or connection may aid to counteract the forces exerted on the steering ring 332 when the operator manipulates or maneuvers one or more pull wires 230 to steer, navigate, articulate and/or position the balloon catheter 218 during cryoablation procedures.

In certain embodiments, as shown in this FIG. 3, the notches 342F, 342S, may be formed, located or positioned on or within the side surface 340 of the steering ring 332. In alternative embodiments, any number of notches 342F, 342S, may be formed, located or positioned on or within the side surface 340. Further, in certain embodiments, as shown herein, the notches, 342F, 342S, may be spaced apart at approximately 180 degrees along the side surface 340. Alternatively, the notches 342F, 342S, may be spaced apart at any suitable distance or pattern.

In certain embodiments, as illustrated in FIG. 3, the notches 342F, 342S, may begin to be formed within the side surface 340 and continue to an opening at the first edge surface 338F. In alternative embodiments, the notches 342F, 342S, may begin to be formed within the side surface 340 and continue to an opening at the second edge surface 338S. In various embodiments, the notches 342F, 342S, may include varying sizes and/or shapes. In some embodiments, the notches 342F, 342S, may have a substantially similar size and/or shape. In other embodiments, the notches, 342F, 342S, may have varying sizes and/or shapes.

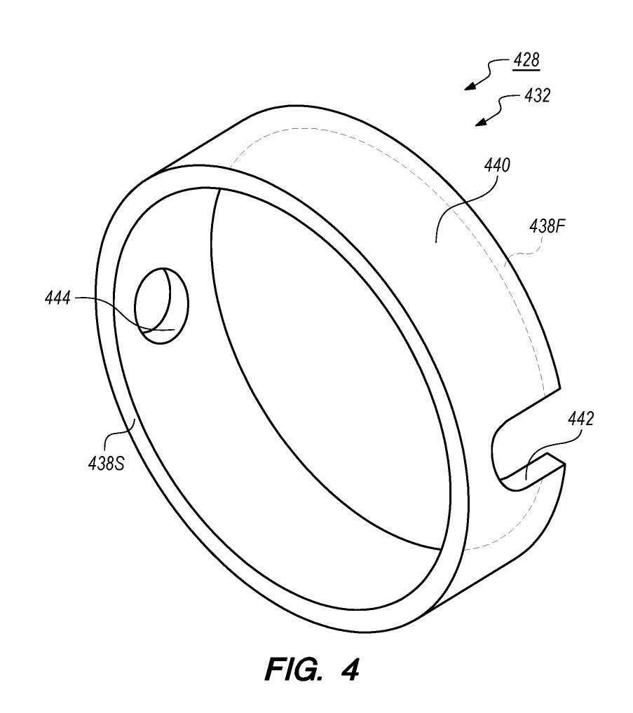

FIG. 4 is a perspective view of still another embodiment of a portion of the steering assembly 428 including another embodiment of the steering ring 432. As shown in the embodiment illustrated in FIG. 4, the steering ring 432 is somewhat similar in shape and/or design to FIG. 3, except that this embodiment includes a singular notch 442 and a singular aperture 444. While FIG. 4 shows the singular notch 442 and the singular aperture 444, as one non-exclusive embodiment, it should be appreciated that the steering ring 432 may include any number of notches 442 (including at least one) and any number of apertures 444.

In the embodiment illustrated in FIG. 4, the steering ring 432 also includes the first edge surface 438F, the second edge surface 438S and the side surface 440. In certain embodiments, the notch 442 and the aperture 444 may be formed, located or positioned on or within the side surface 440 of the steering ring 432. In alternative embodiments, any number of notches 442 and apertures 444 may be formed, located or positioned on or within the side surface 440. Further, in certain embodiments, as shown in FIG. 4, the notch 442 and the aperture 444 may be spaced apart at approximately 180 degrees along the side surface 440.

Alternatively, the notch 442 and the aperture 444 may be spaced apart at any suitable distance and/or pattern. As illustrated in FIG. 4, the notch 442 may begin to be formed within the side surface 440 and continue with an opening at the first edge surface 438F. Alternatively, the notch 442 may begin to be formed within the side surface 440 and continue with an opening at the second edge surface 438S. In various embodiments, the notch 442 and aperture 444 may include varying sizes and/or shapes. In some embodiments, the notch 442 and the aperture 444 may have a substantially similar size and/or shape. In other embodiments, the notch 442 and the aperture 444 may include varying sizes and/or shapes.

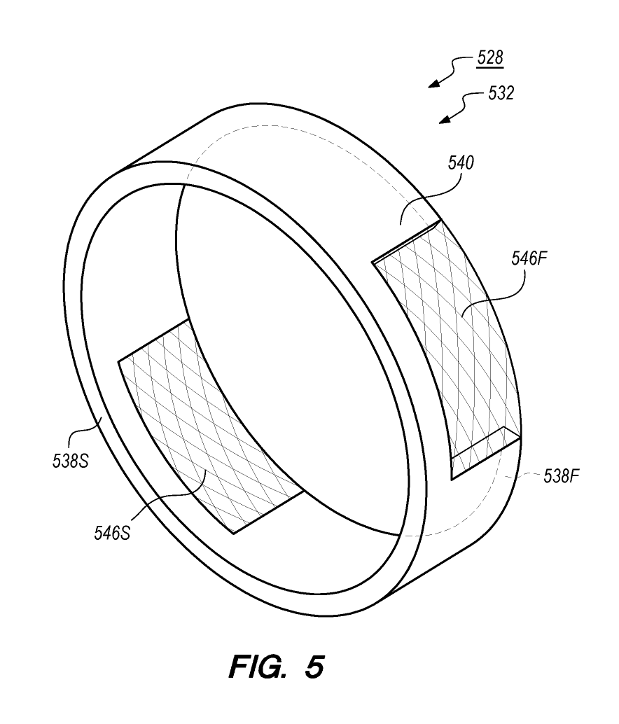

FIG. 5 is a perspective view of yet another embodiment of a portion of the steering assembly 528 including still another embodiment of the steering ring 532. As shown in the embodiment illustrated in FIG. 5, the steering ring 532 is somewhat similar in shape and/or design to FIGS. 3 and 4, respectively, with the exception that this embodiment shows a first lattice structure 546F and a second lattice structure 546S. It is recognized that the terms “first lattice structure 546F” and “second lattice structure 546S” can be used interchangeably. In other words, either lattice structure 546F, 546S, can be the first lattice structure 546F or the second lattice structure 546S.

As referred to herein, the lattice structures 546F, 546S, include the design and/or configuration similar to a stent. The lattice structures 546F, 546S may be formed on or within a portion or area of the side surface 540. While FIG. 5 shows one non-exclusive example of the lattice structures 546F, 546S, it is appreciated that the steering ring 532 may include only one or more than one lattice structures 546F, 546S. In other words, the steering ring 532 can include a plurality of lattice structures 546F, 546S, i.e., first lattice structure, second lattice structure, third lattice structure, etc. Alternatively, the steering ring 532 can also include a singular lattice structure 546F, which in some embodiments may be formed to comprise substantially all or approximately 100% of the side surface 540. Further, while this embodiment shows the lattice structures 546F, 546S, with a design and/or configuration similar to a stent, as one non-exclusive example, it should be appreciated that the lattice structures 546F, 546S, may be of any suitable design, shape, configuration and/or pattern.

In the embodiment illustrated in FIG. 5, the steering ring 532 includes the first edge surface 538F, the second edge surface 538S and the side surface 540. In certain embodiments, the lattice structures 546F, 546S, may be formed, located or positioned on or within the side surface 540 of the steering ring 532. In alternative embodiments, any number of the lattice structures 546F, 546S, may be formed, located or positioned on or within the side surface 540. Further, in certain embodiments, the lattice structures 546F, 546S, may be spaced apart at approximately 180 degrees along the side surface 540. Alternatively, the lattice structures 546F, 546S, may be spaced apart at any suitable distance and/or pattern. As shown in FIG. 5, the lattice structures 546F, 546S, may begin to be formed within the side surface 540 and continue with an opening at the first edge surface 538F. Alternatively, the lattice structures 546F, 546S, may begin to be formed within the side surface 540 and continue with an opening at the second edge surface 538S. Additionally, or alternatively, the lattice structures 546F, 546S, may be positioned at any location on the side surface 540 of the steering ring 532.

In certain embodiments, the lattice structures 546F, 546S, may be contained within a rectangular shape and/or design and formed within the side surface 540. Alternatively, the lattice structures 546F, 546S, may be contained within any suitable shape and/or design of any size. In various embodiments, the steering ring 532 may include one or more lattice structures 546F, 546S. The portion or area of the side surface 540 of the steering ring 532 that contains the one or more lattice structures 546F, 546S can be varied. For example, in some embodiments, the one or more lattice structures 546F, 546S, can comprise at least approximately 10% and less than approximately 80% of the side surface 540 of the steering ring 532. More particularly, in various alternative embodiments, the one or more lattice structures 546F, 546S can comprise at least approximately 10%, 15%, 20%, 25%, 30%, 35%, 40%, 45%, 50%, 55%, 60%, 65%, 70%, 75% or 80% of the side surface 540 of the steering ring 532. Alternatively, the one or more lattice structures 546F, 546S, can comprise greater than 80% or less than 10% of the side surface 540 of the steering ring 532. In one embodiment, a singular lattice structure, i.e., the first lattice structure 546F, may comprise substantially all or approximately 100% of the side surface 540.

In various embodiments, the one or more lattice structures 546F, 546S, may allow the adhesive or bonding material, as non-exclusive examples, to run into and through the lattice structures 546F, 546S, which may aid to counteract the forces exerted on the steering ring 532 when the operator manipulates or maneuvers one or more pull wires 230 (illustrated in FIG. 2) to steer, navigate, articulate and/or position the balloon catheter 218 (illustrated in FIG. 2).

It is understood that although a number of different embodiments of the steering assembly including the steering ring for navigation of the catheter have been illustrated and described herein, one or more features of any one embodiment can be combined with one or more features of one or more of the other embodiments, provided that such combination satisfies the intent of the present invention.

While a number of exemplary aspects and embodiments of the steering assembly including the steering ring for navigation of the catheter have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A steering assembly for a cryogenic balloon catheter system, the steering assembly comprising:
a steering ring having a first edge surface, a second edge surface and a side surface that extends between the first edge surface and the second edge surface, the steering ring including a first notch formed within the side surface, the first notch extending to one of the first edge surface and the second edge surface.

2. The steering assembly of claim 1 further comprising a second notch formed within the side surface, the second notch being spaced apart from the first notch.

3. The steering assembly of claim 2 wherein the second notch is positioned approximately 180 degrees along the side surface from the first notch.

4. The steering assembly of claim 2 wherein the second notch extends to one of the first edge surface and the second edge surface.

5. The steering assembly of claim 2 wherein the first notch and the second notch both extend to the first edge surface.

6. The steering assembly of claim 1 further comprising an aperture formed within the side surface, the aperture being spaced apart from the first notch.

7. The steering assembly of claim 6 wherein the aperture is positioned approximately 180 degrees along the side surface from the first notch.

8. The steering assembly of claim 1 further comprising one or more pull wires that are connected to the steering ring.

9. A steering assembly for a cryogenic balloon catheter system, the steering assembly comprising:
a steering ring having a first edge surface, a second edge surface and a side surface that extends between the first edge surface and the second edge surface, the steering ring including a first lattice structure formed within the side surface.

10. The steering assembly of claim 9, wherein the first lattice structure formed within the side surface extends to one of the first edge surface and the second edge surface.

11. The steering assembly of claim 9 further comprising a second lattice structure formed within the side surface, the second lattice structure being spaced apart from the first lattice structure.

12. The steering assembly of claim 11 wherein the second lattice structure is positioned approximately 180 degrees along the side surface from the first lattice structure.

13. The steering assembly of claim 11 wherein the second lattice structure formed within the side surface extends to one of the first edge surface and the second edge surface.

14. The steering assembly of claim 11 wherein the first lattice structure and second lattice structure both extend to the first edge surface.

15. The steering assembly of claim 11 wherein at least one of the first lattice structure and the second lattice structure formed within the side surface comprises at least approximately 10 percent of the side surface.

16. The steering assembly of claim 11 wherein at least one of the first lattice structure and the second lattice structure formed within the side surface comprises at least approximately 25 percent of the side surface.

17. The steering assembly of claim 11 wherein each of the first lattice structure and the second lattice structure formed within the side surface comprises at least approximately 10 percent of the side surface.

18. The steering assembly of claim 11 wherein each of the first lattice structure and the second lattice structure formed within the side surface comprises at least approximately 25 percent of the side surface.

19. The steering assembly of claim 9 wherein the first lattice structure formed within the side surface comprises at least approximately 50 percent of the side surface.

20. A steering assembly for a cryogenic balloon catheter system, the steering assembly comprising:
one or more pull wires; and
a steering ring connected to the one or more pull wires, the steering ring having a first edge surface, a second edge surface and a side surface that extends between the first edge surface and the second edge surface, the steering ring including a first notch spaced apart from a second notch that are each formed within the side surface, wherein each of the first notch and the second notch extends to one of the first edge surface and the second edge surface.
